(19) United States
(12) Patent Application Publication
(10) Pub. No.: US 2007/0106131 A1
(43) Pub. Date: May 10, 2007

(54) Physiological signal monitoring apparatus for measuring physiological signals of a living subject

(76) Inventors: Yen-Shan Lin, Taipei City (TW); Shing-Huang Tu, Bade City (TW); Ling-Yuan Chou, Hsinchu (TW); Su-Chen Hsu, Zhong-ho city (TW)

Correspondence Address: BRUCE H. TROXELL;SUITE 1404, 5205 LEESBURG PIKE, FALLS CHURCH, VA, 22041, US

(21) Appl. No.: 11/267,499

(22) Filed: Nov. 7, 2005

(51) Int. Cl. A61B 5/00

(52) U.S. Cl. 600/301

(57) ABSTRACT

A health care system includes a health care provider and a physiological signal monitoring apparatus communicated with the health care provider via a network. The monitoring apparatus includes a physiological signal measuring module for measuring the physiological records of a living subject, and a transmission interface for transmitting the measured physiological records to the health care provider.

### FIELD OF THE INVENTION

The present invention relates to a health care system, more particularly to a health care system that includes a physiological signal monitoring apparatus for transmitting the measured physiological records of a living subject to a health care provider via a network.

### BACKGROUND OF THE INVENTION

Due to rapid advance of transportation technology and health care system, the life span of a human being is prolonged for a relatively long years. According to the UNO (United Nation Organization) report on human-life survey, after 300 years from now, average life span of a person will be 95 years, specifically, a Japanese national can live up to 106 years due to their low-salt diet and environment. As the life span prolongs, there will appear several diseases that may endanger the lives of the aged persons. Some chronic diseases, such as hypertension, heart troubles, cancer of different types, paralysis, diabetes, liver cirrhosis, viral hepatitis and arteriosclerosis are well known today and are the most fatal diseases for the aged persons.

Since precaution is better than cure, a person should go under medical check up once in a year when he is over 40 years of age, in addition to taking care of daily eating habits. A person is in command of his own health. Physician and medical staff stand second in line so long as a person's health is concerned, and the person should listen to and take their professional advice as long as his health is concerned. A blood sugar tester, a blood-pressure gauge, a body scale, a cardiograph instrument, a thermometer and an obesity detector are some of the medical-assisting appliances people can rely on and use in their daily life in order to take down essential records of a person's health.

A physiological signal monitoring apparatus is generally used for measuring physiological records of a living subject (including persons suffering from chronic diseases, pregnant women, children, elder persons and medical care takers). A general rule, a person usually follows is that he or she will write down his or her physiological records daily with the assistance of the abovementioned monitoring apparatus (like a thermometer) and he will supply the records to a physician for reference when the former calls on the latter (see FIG. 1). He will visit a clinic or hospital unless on emergency cases, which he cannot attend to himself, such as a sudden fall leading to bone fracture.

For a busy man, it is somewhat difficult to regularly monitor or measure his physiological records. The larger the physiological records are to be monitored, the more difficult for him to accomplish. For example, in order to check the amount of uric acid, cholesterol and trigylceride contained in the blood of a person, the sample blood for undergoing the test is to be drawn out when his stomach is empty (i.e. without taking food for 10 hours long). Another way of precaution method is to take a routine check up of blood sugar level. The person suffering from diabetes must regularly visit a medical center (like township health care center) every 3 to 6 months so as to have the blood sugar level recorded, thereby eliminating the trouble of taking down the physiological records daily by himself.

On the other hand in Taiwan, a health care system known as “family doctor system” is introduced lately into a dwelling community in order to look after the health of the dwelling people within the community. In case, the physiological records of the dwelling people can be transmitted to the health care provider (like clinic or hospital), the health of the dwelling people will be kept under observation, thereby providing the necessary advice to the dwelling people when required.

### SUMMARY OF THE INVENTION

The object of the present invention is to provide a health care system that includes a physiological signal monitoring apparatus via which physiological records of a living subject can be measured and the measured physiological records are immediately transmitted to a health care provider.

The other object of the present invention is to provide a physiological signal monitoring apparatus for use in a health care system. The monitoring apparatus is capable of transmitting the measured physiological records to the health care provider.

In one aspect of the present invention, a physiological signal monitoring apparatus is provided for use in a health care system, the health care system including a network, a health care provider and the physiological signal monitoring apparatus that is communicated with the health care provider via the network. The physiological signal monitoring apparatus includes: a physiological signal measuring module for measuring physiological records of a living subject, a reminding module for immediately generating warning signals and reminding matters based on the warning signals, and a transmission interface for transmitting the physiological records of the living subject to the health care provider. The reminding matters are used for reminding the living subject to conduct a predetermined performance in order to achieve a corresponding result that is transmitted to the health care provider via the transmission interface.

According a second aspect of the present invention, a physiological signal monitoring apparatus is provided for use in a health care system, the health care system including a network, a health care provider and the physiological signal monitoring apparatus that is communicated with the health care provider via the network. The physiological signal monitoring apparatus includes: a physiological signal measuring module for measuring physiological records of a living subject; a recording unit for recording preset reminding matters and the physiological records of the living subject, wherein the recording unit includes a calendar for recording predetermined time data, a reminding module for immediately generating warning signals and reminding matters based on the warning signals; and a transmission interface for transmitting the physiological records of the living subject to the health care provider. The reminding matters are used for reminding the living subject to conduct a predetermined performance when the warning signals matched with the predetermined time data so as to achieve a corresponding result that is transmitted to the health care provider via the transmission interface.

In a third aspect of the present invention, a health care system is provided to include: a physiological signal monitoring apparatus including a physiological signal measuring module for measuring physiological records of a living subject; a reminding module for immediately generating warning signals and reminding matters based on the warning signals; a health care provider for receiving the physiological records of the living subject in order to look after the living subject; a network; and a transmission interface for transmitting the physiological records of the living subject to the health care provider via the network. The reminding matters are used for reminding the living subject to conduct a predetermined performance so as to achieve a corresponding result that is transmitted to the health care provider via the transmission interface.

### BRIEF DESCRIPTION OF THE DRAWINGS

Other features and advantages of this invention will become more apparent in the following detailed description of the preferred embodiments of this invention, with reference to the accompanying drawings, in which:

FIG. 1 shows a conventional system of transmitting a physiological record to a heath care provider;

FIG. 2 shows a health care system of the present invention; and

FIG. 3 is a block diagram representing a physiological signal monitoring apparatus of the present invention.

### DETAILED DESCRIPTIONS OF THE PREFERRED EMBODIMENTS

A health care system of the present invention includes a health care provider and a physiological signal monitoring apparatus having the transmitting capability. The physiological signal monitoring apparatus is adapted to measure the physiological signals of a living subject (a human being), and is adapted to transmit the measured physiological records (signals) to the health care provider. An example of the health care system is given below for better understanding of the present invention, but the scope of the invention should not be limited thereto.

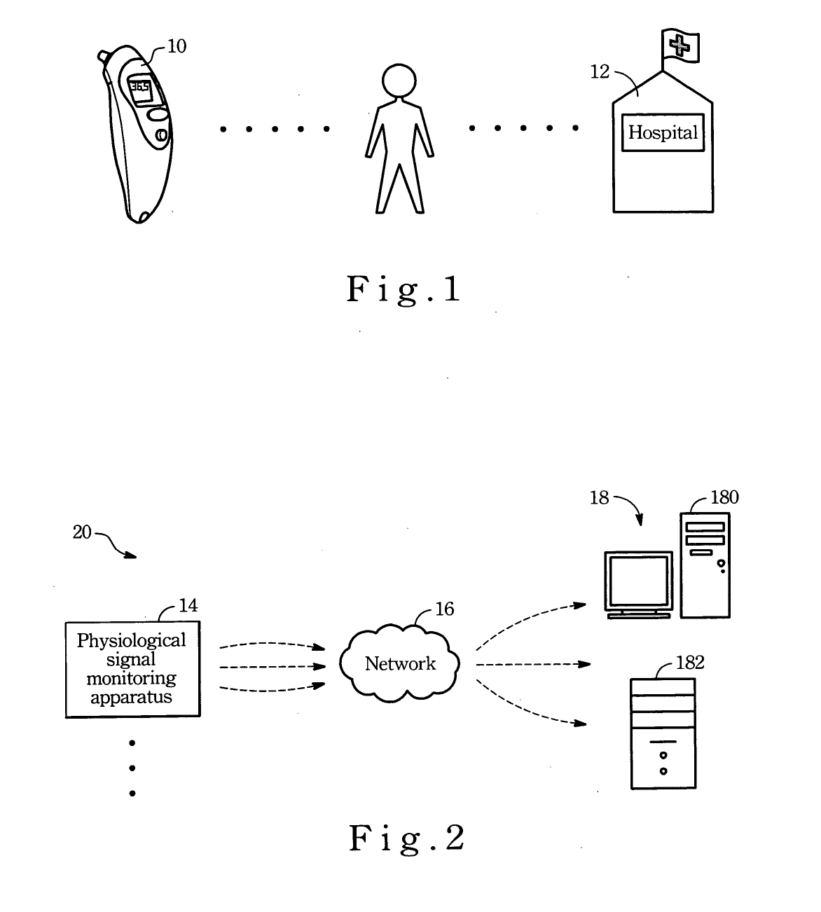

Referring to FIG. 2, the preferred embodiment of a health care system 20 according to the present invention includes a transmission interface (not shown in FIG. 2), a plurality of physiological signal monitoring apparatuses 14 (only one is visible in FIG. 2) and a health care provider 18 that is in communication with the monitoring apparatuses 14 via the network 16. Each physiological signal monitoring apparatus 14 is adapted to measure the physiological records of a living subject (such as measuring blood pressure of a person, conducting blood test to find out the amount of uric acid, cholesterol and trigylceride, testing blood sugar level, taking the temperature, recording down the body weight, liver function, measuring the blood pulse, dimension of a tumor and etc.). The health care provider 18 is adapted to receive the physiological records transmitted by each of the physiological signal monitoring apparatuses 14. The health care provider 18 can be a health care center (such as hospital, clinic and medical foundation). Alternately, the health care provider 18 can be a personal computer 180 or a server 180 (of a dormitory for the elders, security agency and a nursing home), which is communicated with the physiological signal monitoring apparatus 14 via the network 16. The network 16 can be a cable network or a wireless network so long as it can establish communication between the health care provider 18 and the monitoring apparatus 14 so that the transmission interface can transmit the measured physiological records to the medical personnel at the health care provider 18 for reference.

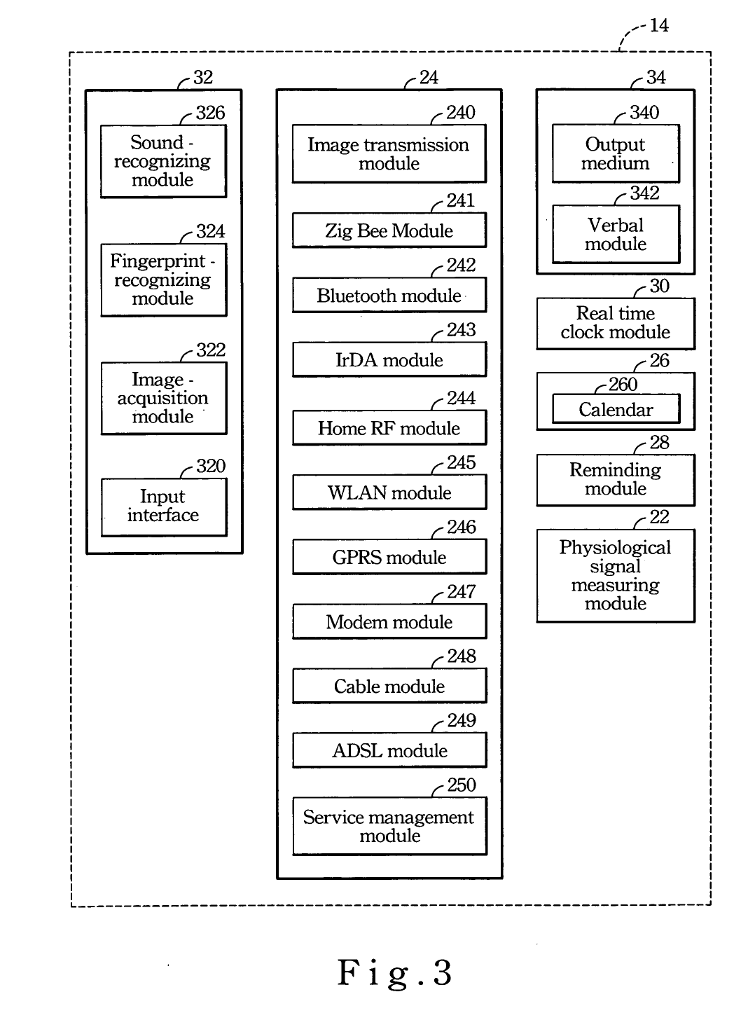

FIG. 3 shows a block diagram representing the physiological signal monitoring apparatus 14 according to the present invention. The monitoring apparatus 14 includes a physiological signal measuring module 22, a transmission interface 24, a recording unit 26, a reminding module 28, a real time clock module 30, an input medium 32 and an output medium 34. The signal measuring module 22 can be selected from a medical device group consisting of a blood-pressure gauge, a blood sugar test, cardiograph instrument, a thermometer, and a fat detector. The signal measuring module 22 can be other medical device so long as it can measure the physiological signals of the living subject.

The transmission interface 24 is used for transmitting the physiological records measured by the aforesaid signal measuring module 22 to the health care provider 18 via the cable network (including wireless network). The transmission interface 24 may include a data transmission module, and can be selected from a wireless module group consisting of a ZigBee module 241, a bluetooth module 242, an IrDA module 243, a home RF module 244, a GPRS module, a WLAN/WiMax module 245 and a GPRS module 246.

In case, the cable network is selected, the user can use a modem module 247, a cable module 248, an ADSL module 249 or a service management module 250. Note that the wireless network structure is preferred only when the distance between the health care provider 18 and the physiological signal monitoring apparatus 14 is relatively short. If the wireless network structure is chosen for transmission of information, prior to transmission of the measured physiological records to the health care provider, a backup file of the physiological records should be recorded in the personal computer, a smart phone or a personal digital assistance for precaution sake and to prevent loss of the measured physiological records of the living subject.

The service management module 250 uses an independent telephone line for transmitting and registering the measured physiological records to the health care provider. Alternately, the service management module 250 may include at least two telephone lines, one of which is used for contacting the health care provider such that the latter can register the transmitted data into the data base under a specific name after identifying and assuring the user's name. Once thus arranged, the health care provider 18 will use the other telephone line to fetch the latest physiological records of the user at preset time intervals.

The recording unit 26 is used for recording preset reminding matters, and includes a calendar for recording predetermined time data. The reminding matters can be light beams of specific color, sound of specific amplitude, letters and calligraphy, warning signs or images. The reminding module 28 can generate the alarm signals and the reminding matters immediately based on the alarm signals. When the alarm signal generated by the reminding module 28 matches with the predetermined time data stored in the calendar, the transmission interface 34 will display the reminding matters, thereby urging and reminding the living subject to conduct a predetermined performance so as to achieve a corresponding result that is transmitted to the health care provider 18 by the transmission interface 34.

An important to note that the alarm signals in the preferred embodiment is produced by the real time clock module 30. The alarm signals are supplied into the reminding module 28 for conducting the matching operation. The recording unit 26 is capable of supplying the physiological records measured at the predetermined time intervals for taking care of the living subject. The reminding module 28 can remind the living subject according to the instructions of the health care provider 18 to conduct the predetermined performance that may include actions of measuring physiological signal of the living subject, taking medicines regularly, calling on the health care provider 18 upon an appointment date, and undergoing rehabilitation exercises or a combination of two actions.

The physiological signal monitoring apparatus 14 of the present invention further includes an input interface 32 via which plurality sets of preset information can be inputted into the transmission interface 24 for transmission to the health care provider 18. The preset information includes the individual physiological records, the reminding matters based on the alarm signals and etc. The input interface 32 includes an input medium 320, such as a sound-recognizing module 326 for recognizing individual sound of the living subject, a finger-print recognizing module 324 for recognizing an individual finger print of the living subject or an image acquisition module 322 for recognizing the mage of the living subject. The image acquisition module 322 may include an image transmission module 240 for processing the image data. The input medium 320 also includes a keyboard and a writing pad for input of codes and letters for transmitting the same to the health care provider 18 in an e-mail form.

The image acquisition module 322 includes optical instruments, such as CCD (charge coupled device) and CMOS (complementary metal-oxide semiconductor). For those persons who have to spend most of the time in bed due to weakness or unable to walk, the finger-print recognizing module 324 is a choice for identifying the individual person when transmitting the measured physiological records the health care provider. In addition, the sound-recognizing module 326 is another alternative for those persons who are short of sight and who are unable to work freely by themselves.

The physiological signal monitoring apparatus 14 of the present invention further includes an output interface 34 having an output medium 340 for displaying the warning signals. The output medium 340 includes a display screen, an amplifier, and a verbal module 342 for broadcasting a procedure for measuring the physiological records of the living subject. When required, the verbally explained procedure can be downloaded and is recorded in the recording unit 26 of the physiological signal monitoring apparatus 14 of the present invention. The recorded procedure can be broadcasted over the amplifier when circumstance required for benefit of the operator nearby.

Some of the benefits provided by the physiological signal monitoring apparatus of the present invention are as follow:
(1) when the physiological signal monitoring apparatus is used, the user is free from the trouble of noting down the measured physiological records (such as blood sugar level, blood pressure) in paper charts, since the monitoring apparatus itself is capable of immediately transmitting the measured physiological records to the health care provider;
(2) the user's life can be saved in case of emergency, since the health care provider can supply medical advice to the user;
(3) the expense for hiring the nursing personnel for looking after the disabled elders can be reduced, since reminding matters and warning signals can eliminate accidents for the disable elders; and
(4) the monitoring apparatus is compatible to the family health care system, since the user's physiological records are kept under observation at the health care provider side without relying on physical paper handling.

While the invention has been described in connection with what is considered the most practical and preferred embodiments, it is understood that this invention is not limited to the disclosed embodiments but is intended to cover various arrangements included within the spirit and scope of the broadest interpretation so as to encompass all such modifications and equivalent arrangements.

I claim:

1. A physiological signal monitoring apparatus for use in a health care system, the health care system including a network, a health care provider and the physiological signal monitoring apparatus that is communicated with the health care provider via the network, the physiological signal monitoring apparatus comprising:
a physiological signal measuring module for measuring physiological records of a living subject;
a reminding module for immediately generating warning signals and reminding matters based on said warning signals; and
a transmission interface for transmitting said physiological records of said living subject to the health care provider;
wherein, said reminding matters are used for reminding said living subject to conduct a predetermined performance in order to achieve a corresponding result that is transmitted to the health care provider via said transmission interface.

2. The physiological signal monitoring apparatus according to claim 1, wherein said predetermined performance includes actions of measuring physiological signals of said living subject, taking medicines regularly, calling on the health care provider upon an appointment date, and undergoing rehabilitation exercises.

3. The physiological signal monitoring apparatus according to claim 1, wherein said warning signals are generated at the health care provider.

4. The physiological signal monitoring apparatus according to claim 1, further comprising a recording unit for recording said physiological records of said living subject and preset reminding matters, said recording unit including a calendar for recording predetermined time data, wherein said reminding module generates said warning signals after comparing said physiological records with respect to said predetermined time data stored in said calendar.

5. The physiological signal monitoring apparatus according to claim 1, wherein said reminding module includes a real time clock module, said alarm signal being generated by said real time clock module.

6. A physiological signal monitoring apparatus for use in a health care system, the health care system including a network, a health care provider and the physiological signal monitoring apparatus that is communicated with the health care provider via the network, the physiological signal monitoring apparatus comprising:
a physiological signal measuring module for measuring physiological signals of a living subject;
a recording unit for recording preset reminding matters and said physiological signals of said living subject, wherein said recording unit includes a calendar for recording predetermined time data;
a reminding module for immediately generating warning signals and reminding matters based on said warning signals; and
a transmission interface for transmitting said physiological signals of said living subject to the health care provider;
wherein, said reminding matters are used for reminding said living subject to conduct a predetermined performance when said warning signals matched with said predetermined time data so as to achieve a corresponding result that is transmitted to the health care provider via said transmission interface.

7. The physiological signal monitoring apparatus according to claim 6, wherein the health care provider is a personal computer and/or a web server.

8. The physiological signal monitoring apparatus according to claim 6, wherein said transmission interface is selected from a module group consisting of a ZigBee module, a bluetooth module, a home RF module, a GPRS module, a WLAN/WiMax module, modem module, an ADSL module, and a service management module 250.

9. The physiological signal monitoring apparatus according to claim 6, wherein said warning signals includes light beams of specific color, a sound of specific amplitude, letters and calligraphy, warning signs or images.

10. The physiological signal monitoring apparatus according to claim 6, further comprising an input medium via which pluralities sets of preset information are transmitted to the health care provider.

11. The physiological signal monitoring apparatus according to claim 10, wherein said input medium includes a sound-recognizing module for recognizing individual sound of said living subject in order to measure said physiological signals.

12. The physiological signal monitoring apparatus according to claim 10, wherein said input medium includes a fingerprint-recognizing module for recognizing an individual fingerprint of said living subject.

13. The physiological signal monitoring apparatus according to claim 10, wherein said input medium includes an image acquisition module for taking a photo of said living subject.

14. The physiological signal monitoring apparatus according to claim 13, wherein said input medium includes an image transmission module for processing image data.

15. The physiological signal monitoring apparatus according to claim 6, wherein said transmission interface further includes an output medium for outputting light signal, sound signal, images and warning signals.

16. The physiological signal monitoring apparatus according to claim 15, wherein said output medium includes a verbal module for broadcasting verbally a procedure for measuring said physiological signals of said living subject.

17. A health care system comprising:
a physiological signal monitoring apparatus including
a physiological signal measuring module for measuring physiological signals of a living subject,
a reminding module for immediately generating warning signals and reminding matters based on said warning signals,
a health care provider for receiving said physiological signals of said living subject in order to look after said living subject,
a network, and
a transmission interface for transmitting said physiological signals of said living subject to said health care provider via said network,
wherein, said reminding matters are used for reminding said living subject to conduct a predetermined performance so as to achieve a corresponding result that is transmitted to the health care provider via said transmission interface.

18. The health care system according to claim 17, wherein said warning signals are generated at said health care provider.

19. The health care system according to claim 17, wherein said predetermined performance includes actions of measuring physiological signals of said living subject, taking medicines regularly, calling on said health care provider upon an appointment date, and undergoing rehabilitation exercises.

20. The health care system according to claim 17, wherein said physiological signal monitoring apparatus includes a recording unit for recording said physiological signals of said living subject and preset reminding matters, said recording unit including a calendar for recording predetermined time data, wherein said reminding module generates said warning signals after comparing said physiological records with respect to said predetermined time data stored in said calendar.

21. The health care system according to claim 17, wherein said physiological signal monitoring apparatus further includes an input medium for inputting said physiological records of said living subject into said transmission interface so that said physiological signals thereof can be transmitted to said health care provider by said transmission interface.

22. The health care system according to claim 21, wherein said physiological signal monitoring apparatus further includes an output medium for displaying said warning signals and outputted signals into said transmission interface such that said corresponding result is transmitted to the health care provider via said transmission interface.

23. The health care system according to claim 17, wherein said care provider is a personal computer and/or a web server.
